(19) United States
(12) Patent Application Publication
(10) Pub. No.: US 2008/0086103 A1
(43) Pub. Date: Apr. 10, 2008

(54) Kit including an absorbent article

(75) Inventors: Robin Lynn McKiernan, Mason, OH (US); Barry Robert Feist, Madeira, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company

(21) Appl. No.: 11/542,782

(22) Filed: Oct. 4, 2006

(51) Int. Cl. A61F 13/15

(52) U.S. Cl. 604/385.02; 600/573; 604/361; 604/385.01; 604/385.06; 206/363; 206/440

(57) ABSTRACT

A kit includes a package, and an absorbent article having a topsheet, a backsheet, and an absorbent core disposed between the topsheet and the backsheet, the absorbent article being disposed in the package in a storage state and removable from the package in an operative state. The kit also may include a supply, such as a diagnostic device, a therapeutic agent, a therapeutic device, or combinations thereof. The supply may be disposed on or in the package in the storage state and detachable from the package in the operative state.

### FIELD OF THE INVENTION

The present disclosure generally relates to a system useful in providing assistance to an infant or child, the system including a supply packaged in combination with an absorbent article, such as a diaper, a pant, or the like.

### BACKGROUND OF THE INVENTION

Any caregiver will recognize that infants and children frequently become ill at the most inopportune times and in the most inopportune locations. Infants and children often show their first or worst symptoms at night, after the infant or child and the caregiver have gone to bed. For that matter, infants and children frequently become ill when the infant or child and caregiver are traveling, for example, on vacation.

When this happens, the caregiver may be ill-prepared to deal with the developing health issue or illness. For example, if the infant or child does not show signs of an illness before the infant or child and the caregiver begin a trip, the caregiver may not pack the necessary supplies, such as cold or fever medication, thermometers, etc., that may assist in diagnosing and treating the illness. Thus, even if a medical professional is available by phone to consult on the illness, the caregiver may be left without the necessary supplies to take full advantage of the consultation. Even if the infant or child and the caregiver are not traveling, the necessary supplies may not be at hand when the infant or child becomes ill. For example, if the caregiver and the infant or child have gone to bed, the supplies, such as a thermometer, may be located in a different part of the house.

On the other hand, it is true that, before the child is toilet trained, it is uncommon for absorbent articles, such as diapers, pants, etc., not to be on hand. While there may be more than one location for changing an infant's diaper in a house, there is typically a diaper-changing station located near the child's bed or sleeping area. Furthermore, even after the child has made the transition to pants, one of the locations where the pants will typically be located is in the child's room. Additionally, if the caregiver and the infant or child are going to travel, it is likely that the caregiver will either pack sufficient numbers of diapers, pants, etc. for the trip, or will plan on purchasing diapers soon upon arrival at the destination.

### SUMMARY OF THE INVENTION

According to an aspect of the present disclosure, a kit includes a package, and an absorbent article having a topsheet, a backsheet, and an absorbent core disposed between the topsheet and the backsheet, the absorbent article being disposed in the package in a storage state and removable from the package in an operative state. The kit also includes a diagnostic device being disposed on the package in the storage state and detachable from the package in the operative state.

According to another aspect of the present disclosure, a kit includes a package, and an absorbent article having a topsheet, a backsheet, and an absorbent core disposed between the topsheet and the backsheet, the absorbent article being disposed in the package in a storage state and removable from the package in an operative state. The kit also includes a supply selected from the group of supplies consisting of a therapeutic agent and a therapeutic device, the supply being disposed on or in the package in the storage state and removable from the package in the operative state.

According to a further aspect of the present disclosure, a kit includes a package, and an absorbent article having a topsheet, a backsheet, and an absorbent core disposed between the topsheet and the backsheet, the absorbent article being disposed in the package in a storage state and removable from the package in an operative state. The kit also includes a supply selected from the group of supplies consisting of a therapeutic agent and a therapeutic device, the supply being disposed on or in the package in the storage state and removable from the package in the operative state, and a diagnostic device being disposed on or in the package in the storage state and removable from the package in the operative state.

### BRIEF DESCRIPTION OF THE DRAWINGS

While the specification concludes with claims particularly pointing out and distinctly claiming the subject matter that is regarded as the present invention, it is believed that the invention will be more fully understood from the following description taken in conjunction with the accompanying drawings. Some of the figures may have been simplified by the omission of selected elements for the purpose of more clearly showing other elements. Such omissions of elements in some figures are not necessarily indicative of the presence or absence of particular elements in any of the exemplary embodiments, except as may be explicitly delineated in the corresponding written description. None of the drawings are necessarily to scale.

FIG. 1 is a perspective view of an embodiment of a kit according to the present disclosure, including a single absorbent article;

FIG. 2 is a perspective view of another embodiment of a kit according to the present disclosure, including a single absorbent article;

FIG. 3 is a perspective view of yet another embodiment of a kit according to the present disclosure, including a single absorbent article;

FIG. 4 is a perspective view of still another embodiment of a kit according to the present disclosure, including a single absorbent article;

FIG. 5 is a perspective view of an embodiment of a kit according to the present disclosure, including a plurality of absorbent articles;

FIG. 6 is a perspective view of another embodiment of a kit according to the present disclosure, including a plurality of absorbent articles;

FIG. 7A is a plan view of an embodiment of a temperature sensor according to an embodiment of the present disclosure;

FIG. 7B is a side view of the temperature sensor of FIG. 7A, with the thicknesses of the constituent layers exaggerated for ease of discussion;

FIGS. 8A and 8B illustrate a change in an embodiment of a temperature sensor utilizing a linguistic indicator;

FIGS. 9A and 9B illustrate a change in an embodiment of a temperature sensor utilizing a graphical indicator;

FIGS. 10A and 10B illustrate a change in an embodiment of a temperature sensor utilizing a color indicator;

FIGS. 11A-11C illustrate a change in an embodiment of a temperature sensor utilizing a plurality of regions and graphical indicators;

FIG. 12 illustrates an alternative embodiment of a temperature sensor utilizing a plurality of regions and numeric indicators;

FIGS. 13A and 13B illustrate a change in an embodiment of a temperature sensor utilizing a band of colors;

FIG. 14 illustrates an embodiment of a temperature sensor formed with an absorbent article;

FIG. 15 illustrates an embodiment of a temperature sensor formed with an absorbent article, the temperature sensor being detachable from the absorbent article;

FIG. 16 is a plan view of an exemplary absorbent article, with a section of a topsheet removed to expose an underlying absorbent core; and

FIG. 17 is a perspective view of the absorbent article of FIG. 16 shown in its relaxed, contracted state, i.e., with the contraction induced by elastic members.

### DETAILED DESCRIPTION OF THE INVENTION

### DEFINITIONS

As used herein, the following terms have the following meanings:

The term “absorbent article” refers to a device that absorbs and contains liquid, and more specifically, refers to a device that is placed against or in proximity to the body of the wearer to absorb and contain the various exudates discharged from the body.

The terms “adhesively bonded” or “adhesively laminated” refers to a laminate wherein an adhesive is used to bond the elastomer member to the nonwoven or nonwovens.

The term “attached” refers to elements being connected or united by fastening, adhering, bonding, etc. by any method suitable for the elements being attached together and their constituent materials. Many suitable methods for attaching elements together are well-known, including adhesive bonding, pressure bonding, thermal bonding, mechanical fastening, etc. Such attachment methods may be used to attach elements together over a particular area either continuously or intermittently.

The term “caregiver” refers to a person other than the child, such as, a parent, babysitter, family member, day care worker, or other person who is able to provide assistance to the child to complete a personal task.

The term “diaper” refers to an absorbent article generally worn by infants and incontinent persons about the lower torso and having the general form of a sheet, different portions of which are fastened together to encircle the waist and the legs of the wearer.

The term “disposable” refers to absorbent articles that generally are not intended to be laundered or otherwise restored or reused as absorbent articles, i.e., they are intended to be discarded after a single use and, preferably, to be recycled, composted or otherwise disposed of in an environmentally compatible manner.

The term “disposed” is used to mean that an element(s) is formed (joined and positioned) in a particular place or position as a unitary structure with other elements or as a separate element joined to another element.

The terms “interior” and “exterior” may refer respectively to the location of an element that is intended to be placed against or toward the body of a wearer when an absorbent article is worn and the location of an element that is intended to be placed against or toward any clothing that is worn over the absorbent article. “Interior” and “exterior” may also refer to a particular orientation of elements relative to each other, without specific or special reference to the wearer. Synonyms for “interior” and “exterior” include, respectively, “inner” and “outer”, as well as “inside” and “outside”. Also, when an absorbent article is oriented such that its interior faces upward, for example, when it is laid out in preparation for setting the wearer on top of it, synonyms include “upper” and “lower” and “top” and “bottom”, respectively.

The term “joined” refers to configurations whereby an element is directly secured to another element by attaching the element directly to the other element, and configurations whereby an element is indirectly secured to another element by attaching the element to intermediate member(s) which in turn are attached to the other element.

The term “lateral” or “transverse” refers to a direction running at a 90 degree angle to the longitudinal direction and includes directions within ±45° of the lateral direction.

The term “longitudinal” refers to a direction running parallel to the maximum linear dimension of the article and includes directions within ±45° of the longitudinal direction.

The term “macroporous” refers to materials having pores too large to effect capillary transport of fluid, generally having pores greater than about 0.5 mm in diameter and, more specifically, having pores greater than about 1.0 mm in diameter. The term “microporous” refers to materials which are capable of Laplace pressures greater in magnitude than approximately 10 kPa.

The term “pant” or “pants” refers to an absorbent article generally worn by infants and incontinent persons about the lower torso and having the general form of a pair of short pants that can be applied or removed from the wearer without unfastening. A pant may be placed in position on the wearer by inserting the wearer's legs into the leg openings and sliding the pant into position about the wearer's lower torso. While the term “pant” is used herein, pants are also commonly referred to as “closed diapers”, “prefastened diapers”, “pull-on diapers”, “training pants”, “diaper-pants”, and “swim pants”.

The term “refastenable” refers to the property of two elements being capable of releasable attachment, separation, and subsequent releasable reattachment without substantial permanent deformation or rupture.

The terms “releasably attached,” “releasably engaged,” and variations thereof refer to two elements being connected or connectable such that the elements tend to remain connected absent a separation force applied to one or both of the elements, and the elements being capable of separation without substantial permanent deformation or rupture. The required separation force is typically beyond that encountered while wearing the absorbent garment.

### Exemplary Kit

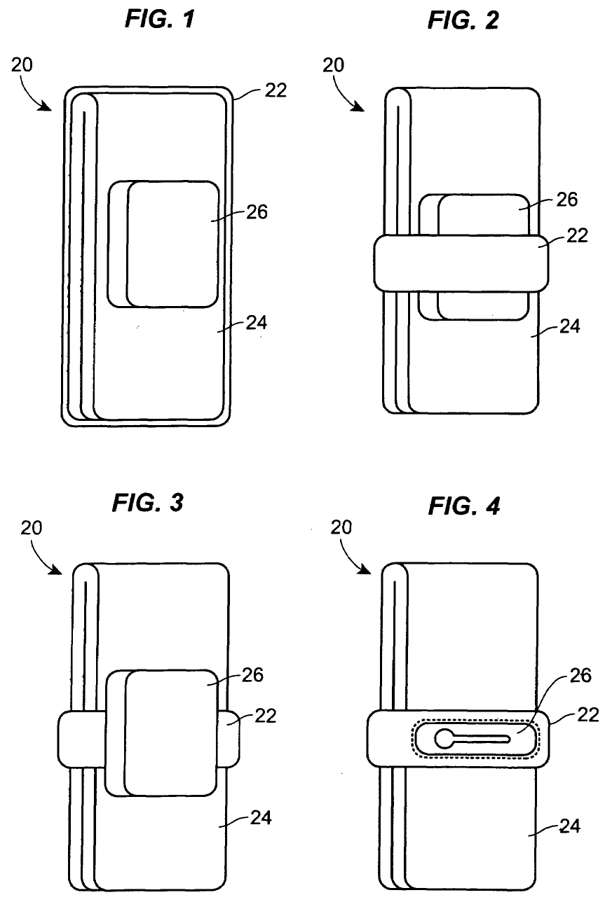

FIGS. 1-6 illustrate embodiments of a kit 20 according to the present disclosure. The system 20 may include a package 22, an absorbent article 24, such as a diaper, pant, etc., and at least one supply 26. The absorbent article 24 is disposed on or in the package in a storage state and removable from the package in an operative state. The supply or supplies 26 is disposed on or in the package in the storage state, and removable from the package in the operative state. As used herein, the storage state may refer to a state wherein the absorbent article or the supply is held in readiness for use. The operative state may refer to a state wherein the absorbent article or the supply may be used by the caregiver or child.

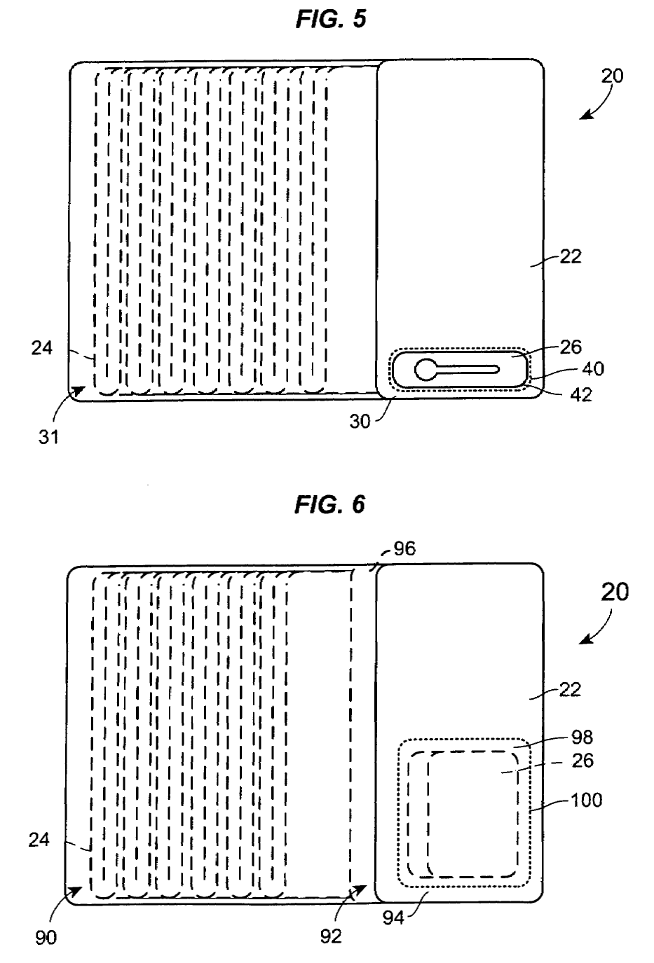

According to the present disclosure, the package 22 may take various forms. As one example, the package 22 may associate a single absorbent article 24 and a single supply 26. According to an embodiment illustrated in FIG. 1, the package 22 may be a plastic “shrink-wrap” container or bag, which is formed about the absorbent article 24 and the supply 26, which package 22 may permit the system 20 to be sold as a unit, for example, with other supplies 26, as opposed to with absorbent articles 24. Alternatively, the package 22 may be a loop or a band of paper, cardboard, or the like, that is fastened about the article 24 and the supply 26 (FIG. 2), or that is fastened about the article 24 and to which the supply 26 is joined (FIG. 3). As another example, the supply 26 may be disposed on the package 22, which may be a loop or a band of paper, cardboard or the like, that is fastened about the article 24 (FIG. 4). As still another example, the package 22 may associate a plurality of absorbent articles 24 and one or more supplies 26 (FIGS. 5 and 6).

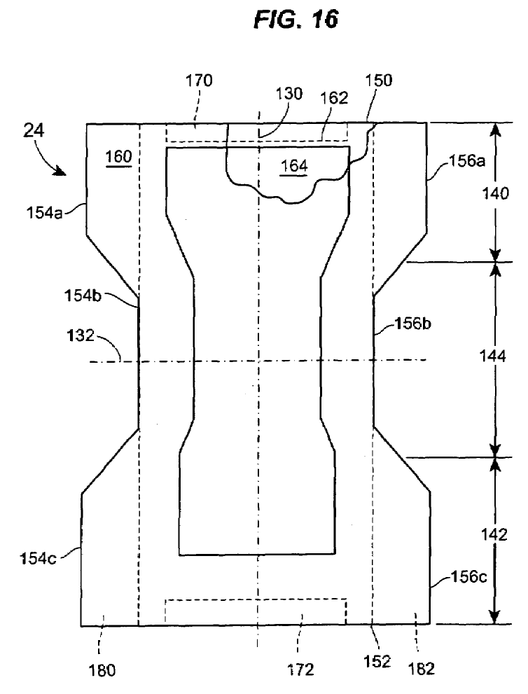

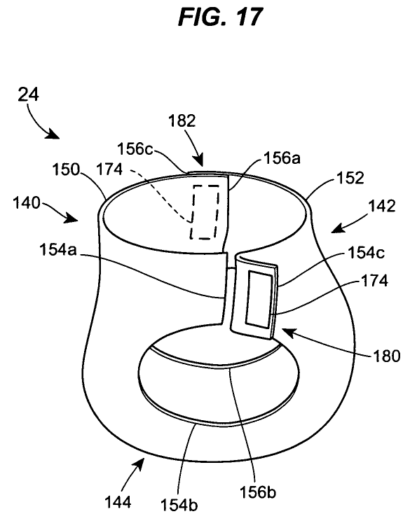

The absorbent article 24 also may take various forms. In general terms, the absorbent article 24 may include a topsheet, a backsheet and an absorbent core disposed between the topsheet and the backsheet. However, as mentioned above, the article 24 may be a diaper, pant, etc. Furthermore, the article may include fasteners, cuffs, etc., the details of which are further discussed below relative to FIGS. 16 and 17.

According to one group of embodiments, the supply 26 may be one that is used by a caregiver to measure a symptom (for example, temperature) useful, for example, in the diagnosis of an illness. Alternatively, the supply 26 may be one that is used by a caregiver to treat an illness or a symptom of an illness.

One example of a supply 26 that is used by a caregiver to measure a symptom useful in the diagnosis of an illness is a diagnostic device. For example, one readily-recognizable diagnostic device is a thermometer, as is illustrated in the embodiments in FIGS. 4 and 5. However, the diagnostic device may include other testing devices, such as pre-packaged test kits for use, for example, in combination with exudates, such as urine.

The diagnostic device may be a “single” or “limited” use device not intended for use over a prolonged period (i.e., more than once or a limited number of times, or more than one period of illness), or may be a “longer-term use” device intended to be useful over a prolonged period. As an example of the former, the thermometer may be defined by a region of temperature-sensitive material disposed on a substrate that may be conveniently disposed of, such as paper or plastic, for example. Alternatively, the diagnostic device may include a sensor, a display (such as a liquid crystal display), a circuit to receive the signal from the temperature sensor and actuate the display so as to display a visible representation of the temperature as determined by the temperature sensor, and a battery to power the circuit and the display. It will be recognized, however, that a temperature sensor defined by a region of temperature-sensitive material disposed on a substrate may be formed in such a way to permit its use over a prolonged period of time (for example, through the use of a sturdy substrate), and a temperature sensor including a sensor, display, circuit and batter may be packaged so as to be disposable after one use, if desired. Thus, there is a considerable degree of latitude in what may be termed a “single” or “limited” use device, and what may be termed a “longer-term use” device.

In either case, the diagnostic device may be packaged with other articles useful with the device, for example, in its operation or cleaning. One example of such other articles would be sleeves, for example, made of plastic, that may be fitted over the device to permit the device to be used with several children with limited likelihood of cross-contamination. An alternative example would be alcohol wipes that may be used to clean the device after use.

One example of a supply 26 that may be used by a caregiver to treat an illness or a symptom of an illness is a therapeutic agent. For example, the therapeutic agent may be a cold or fever medicine. Alternatively, the therapeutic agent may be a useful in treating a particular condition, such as constipation or diarrhea. As another alternative, the therapeutic agent may be useful in relieving heartburn, acid indigestion, sour stomach and upset stomach, such as CHILDREN'S PEPTO calcium carbonate/antacid commercially available from The Procter & Gamble Co. of Cincinnati, Ohio. As a further alternative, the therapeutic agent may be a compound that provides a soothing effect, for example a rub that contains materials such as eucalyptus oil, lavender oil, and rosemary oil, such as VICKS BABYRUB soothing ointment commercially available from The Procter & Gamble Co. of Cincinnati, Ohio.

According to certain examples, the therapeutic agents may be packaged for “single use”—i.e., a limited amount of the compound is disposed in a package to permit a child or infant to be treated once. According to other examples, the therapeutic agents may be packaged in such amounts as to permit the caregiver to provide the therapeutic agent to the child or infant over a prolonged treatment period (i.e., more than once). According to some examples, in particular those examples wherein a sufficient amount of the therapeutic agent is provided for a prolonged treatment period, the therapeutic agent may be packaged in a container that includes a delivery mechanism (cup, syringe, etc.) or may be packaged with a delivery mechanism.

As another example of a supply 26 that may be used by a caregiver to treat an illness or a symptom of an illness may be a therapeutic device. For example, the therapeutic device may be a hot or cold pack or compress. According to such an embodiment, the hot or cold pack may include one or more chemicals that, when mixed together, produce a reaction that is exothermic or endothermic.

As noted above, according to certain embodiments, the supply 26 may be disposed on the package 22. As already mentioned above, where the package 22 is a loop or band that associates a single absorbent article 24 and a single supply 26, the supply 26 may be attached to a surface of the package 22 (see FIG. 3). For example, the supply 26 may be attached to an exterior or an interior surface of a wall of the package 22 by a method such as adhesion, hook and loop fastening, sewing, clamping, magnetism, binding by an outer layer, heat sealing, ultrasonic bonding, and combinations thereof. In certain cases, the supply 26 may be removably fastened, for example, via the hook and loop fastening, reusable adhesives, tab and slot fasteners, snap fasteners, etc. Where adhesives are used, they may be pressure activated adhesives or tapes and heat activated adhesives. According to such an embodiment, the supply 26 may be disposed in its own, separate package, and then attached to the package 22.

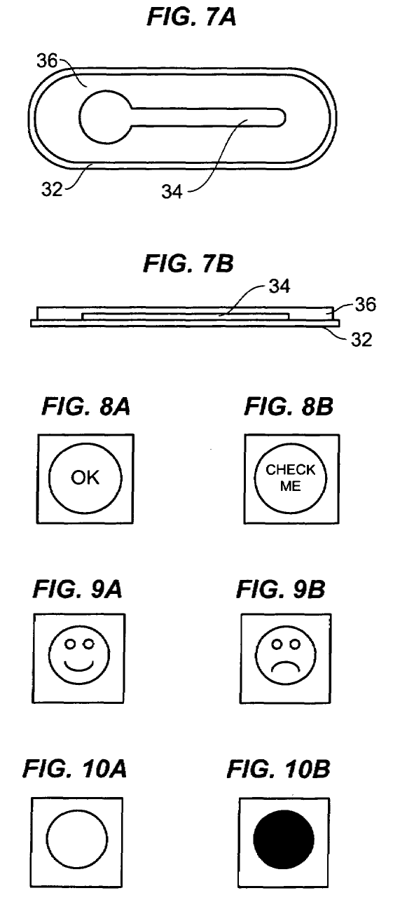

Alternatively, the supply 26 may be defined, at least in part, by the package 22, but be formed in such a manner as to be detachable from the package 22, so as to be disposed on the package. For instance, as is illustrated in FIG. 5, the package 22 may include an outer wall 30, the outer wall 30 defining a space 31 in which one or more disposable absorbent articles 24 may be disposed. According to this embodiment, a plurality of absorbent articles 24 may be disposed in the package 22. The supply 26 may include one or more layers, at least one of which may be defined by the outer wall 30 of the package 22. As illustrated in FIG. 7A and 7B, the supply 26 may be a diagnostic device in the form of a temperature sensor. The temperature sensor 26 may include a substrate layer 32, which is defined by the outer wall 30 of the package 22, on which may be disposed a temperature-sensitive layer 34 (which, despite the name, may include temperature-sensitive or temperature-insensitive materials), these layers 32, 34 in combination defining the temperature sensor 26, or thermometer. Optionally, another layer 36 of material may be disposed over the temperature-sensitive layer 34, which layer 36 may be transparent or translucent, and which layer 36 may act to prevent damage to the temperature-sensitive layer 34, to improve the readability of the sensor 26 through magnification or other optical enhancement effects, or to insulate the temperature-sensitive layer 34 from ambient temperatures to thereby limit the ambient temperatures influence on the sensor 26.

As illustrated in FIG. 5, the package 22 may also include a detachment mechanism, in the form of a perforation 40 that permits a caregiver to detach the temperature sensor 26 from the wall 30 of the package 22. According to certain embodiments, the perforation 40 may define a region 42 that is detachable not only to separate the sensor 26 from the package 22, but to allow access into the package 22 and the article or articles 24 disposed therein. In such an embodiment, the region 42 defined by the perforation 40 may be referred to as a closure element.

The temperature sensor 26, thus defined, may come in a variety of shapes and colors. For example, a shape may be a circle, a partial circle, square, ellipse, rectangle, triangle, elongated strip, a non-geometric shape, or combinations thereof. As to colors, the sensor 26 may be colored to coordinate and fit with the colors and design of the package 22; alternatively, the sensor 26 may be colored to distinguish itself from the remainder of the package 22.

The temperature-sensitive layer 34 of the sensor 26 may include one or more materials selected from the group consisting of thermochromic liquid crystalline materials, thermochromic dyes, thermochromic inks, and combinations thereof. As used herein “thermochromic” means materials/inks that change their reflected color as a function of temperature. In particular, thermochromic dyes that can change color are called lueco dyes and such can be directly mixed in films, nonwovens, and elastics. Lueco dyes are commercially available from HW Sands Corp. of Jupiter, Fla. and Color Change Corp. of Streamwood, Ill. Suitable thermochromic liquid crystalline materials may be either temperature sensitive or temperature insensitive and chiral or cholesteric in nature. Suitable chiral and/or cholesteric thermochromic liquid crystalline materials may be like those incorporated into patches sold by Hallcrest Incorporated of Glenview, Ill., Kaz Inc. of Hudson, N.Y., Liquid Crystal Resources, LLC of Glenview, Ill., Medical Indicators of Pennington, N.Y., and/or Thermographic Measurements of Flintshire, UK. Suitable thermochromic inks may be commercially available from Chromatic Technologies, Inc. under the tradename DYNACOLOR as body temperature or high temperature inks or from Sun Chemical's AIC subsidiary of France under the name THERMASOFT. Additional suitable thermochromic inks are detailed in U.S. Pat. Nos. 4,121,011; 4,826,550; 5,389,093; and 5,221,228. The thermochromic materials used can be in the form of fine pigments, particles, microencapsulated materials, molecular materials and the like.

The one or more materials may be applied according to a method such as spraying, printing, coating, ultraviolet printing, painting, and combinations thereof. Suitable printing methods include, but are not limited to gravure, flexo, inkjet, slot, and screen printing.

In certain instances, the temperature insensitive thermochromic liquid crystalline material is colored at room temperature and normal human body temperatures and changes to clear in appearance in response to a noticeable increase in human body temperature. However, there are other suitable types of thermochromic liquid crystalline materials (temperature sensitive) that turn from colorless to red to orange to yellow to green to blue to violet and then back to colorless as the temperature is increased.

The substrate layer may be printed black to enhance the appearance of the thermochromic liquid crystalline material phase transformations but this substrate layer also may be printed to be colored such that the substrate layer may become visible in the sensor through the thermochromic liquid crystalline material as it becomes transparent or translucent in appearance. Alternatively, a layer may be disposed between the substrate layer and the temperature sensitive layer, and may be defined by a photochromic ink. Photochromic inks change color in response to the presence of ultraviolet or other wavelengths of radiation. In most cases, the photochromic inks change from invisible or clear to a humanly perceivable color upon exposure to a particular range of wavelengths.

As noted above, a layer 36 may be disposed over the temperature-sensitive layer 34. The layer 36, which may be referred to as an overlayment layer, may be at least partially transparent or translucent such that the temperature-sensitive layer 34 is visible through the layer 36. Suitable materials for this layer include polymeric materials, such as polyolefins (polyethylenes, polypropylenes, etc. and combinations thereof), polyesters, etc. and combinations thereof. This overlayment layer 36 may serve to contain or protect the thermochromic liquid crystalline material, serve as a thermoconductive or insulating material for the sensor 26 on the skin of a wearer, or serve as a contributor to enhance the appearance of the indicator that is placed on the face of the sensor 26.

For instance, the overlayment layer 36 may be printed to form one or more indicia. These indicia are selected from the group consisting of linguistic indicia (FIGS. 8A and 8B), graphical indicia (FIGS. 9A and 9B), numerical indicia, color indicia (FIGS. 10A and 10B), and combinations thereof. The numerical indicia might provide an exact numerical read-out of the skin or ambient temperatures. The linguistic indicia provide some written signal to the caregiver that a change in condition has been detected. For instance, a suitable linguistic indicator may include the phrases “check me”, “fever”, “warm me up”, “I'm warm”, “I'm cold”, “check temp”, “check my temperature”, “OK”, etc. For instance, FIGS. 8A and 8B depict a sensor wherein the linguistic indicia changes from “OK” to “CHECK ME” when the requisite temperature change is indicated. A graphical indicia, such as is illustrated in FIGS. 9A and 9B, provides a pictorial depiction that serves as a signal to the caregiver that a change in condition has been detected in the body temperature of the wearer. For example, a graphical indicia envisioned include the appearance of the following images in the event a high temperature is detected by the sensor: a sad or frowning face, a thermometer, a face with a thermometer, a single flame, a checkmark, a bold “X” mark, etc. A hypothermic condition might be graphically suggested with an icicle, blanket or other suitable graphical image. A color indicia, such as is illustrated in FIGS. 10A and 10B, provides a change in color of the sensor that is easily noticeable by a caregiver wherein the change in color appears when an abnormally high or low temperature is detected by the sensor. For instance, the sensor may change from black to green in color, from clear to black, from red to clear, from green to black, etc. It will be recognized that the change in color could also be achieved through the use of color-changing thermochromic material, and a clear overlayerment layer 36.

It may also be recognized that it would be desirable to provide a certain amount of time between the instance of removing the sensor from the skin of the infant or wearer and the instance of reading the body temperature of the wearer from the sensor by the caregiver before the indicator changes as a result of being removed from the wearer's body and influenced by the ambient conditions (i.e., the environment of the wearer and/or user). This can be accomplished through either hysteresis or delay. As used herein, “hysteresis” refers to the characteristic where the transition temperature from a transparent thermochromic liquid crystalline material to a color reflecting thermochromic liquid crystalline material as temperature decreases is lower than the transition temperature from a color reflecting thermochromic liquid crystalline material to a transparent thermochromic liquid crystalline material as temperature increases. The hysteresis characteristic of the material delays the temperature at which the transition from the transparent phase to color reflecting phase occurs. The skin temperature as measured by the thermochromic liquid crystalline material sensor may also be locked for longer periods of time by applying a layer of material to the top, bottom or both regions of the structure. For instance, the sensor may be modified to exhibit a delay in temperature change of about 1 second, 5 seconds, 10, seconds, 30 seconds, 60 seconds, 90 seconds, or even about 120 seconds. Specifically, the top and bottom layers of material should be thermally capacitive, such as an elastomeric material like Exxon's VISTA MAXX with entrained low meltpoint waxes like Licowax PP230 and ChevTex 128 (commercially available from Clariant and Renkert Oil, respectively). These materials are polymers with an entrained low meltpoint wax, capable of absorbing or releasing significant amounts of heat energy before increasing or decreasing in temperature. Suitable polymer materials are disclosed in U.S. applications Ser. Nos. 11/042236 and 11/042237, both filed on Jan. 25, 2005 in the name of J. P. Autran and both entitled “Fibers and Nonwovens Comprising Polypropylene Blends.”

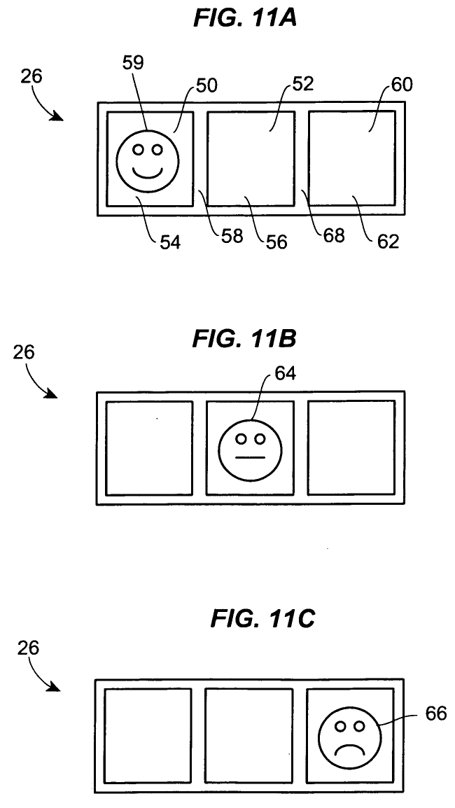

In a particular embodiment, illustrated in FIGS. 11A, 11B, and 11C, the sensor 26 comprises at least a first and a second material 50, 52, whether thermochromic ink, thermochromic dye or thermochromic liquid crystalline material, in a respective first and second measurement region 54, 56 wherein the first and second materials 50, 52 are physically separated from one another within the sensor 26 by a separator 58. It is envisioned that there may be additional materials 60 and corresponding measurement regions 62. Such an embodiment could be useful in the instance where, for example, a first measurement region 54 comprising the first thermochromic liquid crystalline material 50 was positioned toward the left end of the sensor 26. This first material 50 turns a particular color, for example, green, at normal human body temperatures (for example, 96° F. to 100° F.), and may be overlaid with an overlayment that is printed with a graphical or linguistic indicator 59. The second material 52 would be positioned toward the middle of the sensor 26 in the second measurement region 56. This second material 52 changes color at temperatures greater than about 100° F. and the corresponding area 56 of the sensor 26 that it covers is then further overlaid with an overlayment that is printed with a graphical or linguistic indicator 64. Additional measurement regions 62 within the sensor 26 may be carved out as well that include additional thermochromic liquid crystalline materials, thermochromic inks, thermochromic dyes or combinations thereof. For instance, the above-described sensor 26 may include a third thermochromic liquid crystalline material 60 that changes color at temperatures greater than about 101° F. This third area 62 in the sensor 26 may comprise a similar overlayment layer that is printed with an indicator 66 to signal the caregiver of a fever condition in the wearer. The areas 54, 56, 62 of the sensor 26 are distinguished from one another by one or more separators 58, 68 that serve as barriers between the different types of thermochromic liquid crystalline materials 50, 52, 60 within the sensor 26.

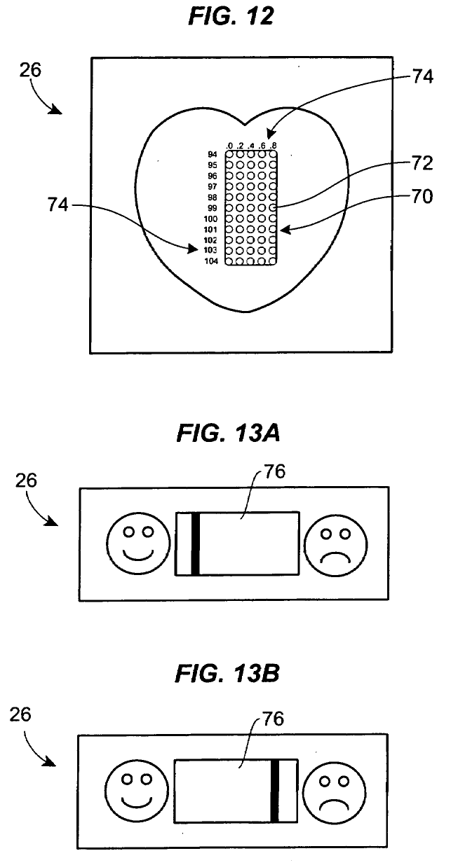

Another embodiment of a sensor 26 with a plurality of regions is illustrated in FIG. 12. According to this embodiment, the sensor 26 includes a plurality of measurement regions 70 that are physically separated from each other. Each of the measurement regions 70 includes a material 72, such as a thermochromic ink, thermochromic dye, or thermochromic liquid crystalline material. The materials may be selected so as to permit a range of temperatures to be detected, and to permit the temperatures to be discerned to within a range of precision. So that the caregiver may interpret the results, the sensor 26 also includes information, in the form alphanumeric characters, for example, displayed in certain areas 74 of the sensor 26. As illustrated, the sensor is may be useful over a range of temperatures from 94-105° F., in increments of 0.2° F. The range of precision is also ±0.2° F., insofar as it is not possible to discern gradations in temperature of less than 0.2° F.

Still another embodiment that is envisioned within the scope of this disclosure is the incorporation of a moving line thermochromic liquid crystalline material sensor, as illustrated in FIGS. 13A and 13B. The moving line thermochromic liquid crystalline sensor 26 is a band 76 of multiple colors that moves across a scale as the detected temperature changes. For instance, the band 76 of color may traverse the visible light spectrum with the leading edge usually violet and the trailing edge usually red in color or vice versa with the leading edge being red and the trailing edge being violet. Alternatively, the band 76 of color may exhibit other color combinations. The most effective background to enhance the visibility of the band 76 of colors is black although other background colors can be used. The band 76 of color can be designed to move vertically, horizontally, or at any angle in between. As the temperature increases, the band 76 can move from lower to high on the appropriate axis.

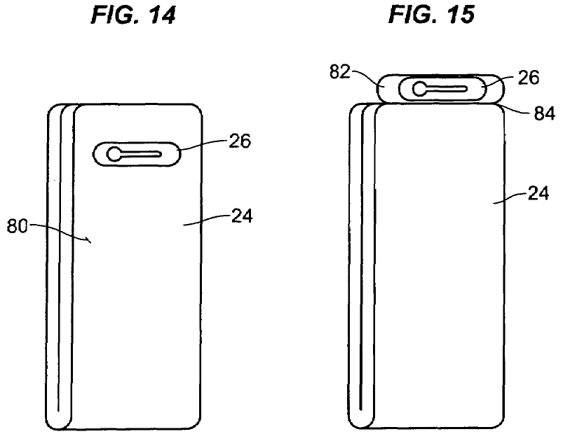

Still further embodiments are illustrated in FIGS. 14 and 15. According to these embodiments, a sensor 26 is formed with the article 24. For instance, in FIG. 14, the sensor 26 is formed on a surface 80 of the article 24. Alternatively, in FIG. 15, the sensor is formed on a tab or flap 82 that is attached to the article 24. A perforation 84 may be defined along an interface between the tab or flap 82 and the remainder of the article 24, such that the sensor 26 may be used in place by folding the tab or flap 82 over, thereby placing the sensor 26 against the skin of the infant or child, or may be used by detaching the sensor from the article 24 and used separately. Further details of an article with a sensor that is formed with the article can be found in U.S. Patent Application No. 60/756,237, filed Jan. 3, 2006.

In the alternative to disposing the supply 26 on the package 22, the supply 26 may be disposed in the package 22. According to some embodiments, such as one illustrated in FIG. 1, the package 22 may be a shrink-wrap package 22 associating a single article 24 and the supply 26 disposed within the package 22. According to other embodiments, the supply 26 may be disposed within its own packaging, and then disposed within the package 22 with the article or articles 24.

According an embodiment illustrated in FIG. 6, the package 22 may have two compartments: a first compartment 90 in which the absorbent articles 24 are disposed and a second compartment 92 in which the supply 26 is disposed. According to such an embodiment, the package 22 may have a first outer wall 94, that limits access to the articles 24 and the supply 26, and a second inner wall 96 (which may or may not be joined to the outer wall 94), that divides a space enclosed by the outer wall 94 into the two compartments 90, 92. The outer wall 94 may have a closure element 98, defined by a perforation 100 in the outer wall 94, that permits access to the articles 24, but not the supply 26, or vice versa.

It will also be recognized that any of the embodiments above may illustrate an embodiment wherein more than one supply 26 is disposed on or in the package 22. As one example, where the supplies 26 are disposed in a package that is then joined to the package 22, it will be recognized that the package for the supplies 26 could include more than one type of supply, for example a diagnostic device and a therapeutic agent. Alternatively, where the diagnostic device is formed with the article 24, as illustrated in FIGS. 14 or 15, it will be understood that a diagnostic device may be packaged with another supply 26 simply by packaging an article 24 as illustrated in FIGS. 14 and 15 with the other supply 26, such as is illustrated in FIGS. 1-3 or 6, for example. For that matter, the embodiments of FIGS. 5 and 6 may be combined to provide an embodiment wherein a diagnostic device and another supply 26 are combined with articles 24 and a package 22.

### Exemplary Absorbent Article

Having been discussed in general terms above, the absorbent article 24 is now discussed in greater detail with reference to FIGS. 16 and 17.

FIG. 16 is a plan view of an exemplary disposable absorbent article 24 in its flat, uncontracted state, i.e., without elastic-induced contraction. Portions of the article 24 have been cut away to more clearly show the underlying structure of the disposable absorbent article 24. As illustrated, the portion of the disposable absorbent article 24 that contacts the wearer faces the viewer (i.e., showing the interior or inner side of the article). The disposable absorbent article 24 has a longitudinal axis 130 and a transverse axis 132.

One end portion of the disposable absorbent article 24 is configured as a first waist region 140 of the disposable absorbent article 24. The opposite end portion is configured as a second waist region 142 of the disposable absorbent article 24. The waist regions 140 and 142 generally comprise those portions of the disposable absorbent article 24 which, when worn, encircle the waist of the wearer. The waist regions 140 and 142 may include elastic elements such that they gather about the waist of the wearer to provide improved fit and containment. An intermediate portion of the disposable absorbent article 24 is configured as a crotch region 144, which extends longitudinally between the first and second waist regions 140 and 142. The crotch region 144 is that portion of the disposable absorbent article 24 which, when the disposable absorbent article 24 is worn, is generally positioned between the legs of the wearer.

The disposable absorbent article 24 has a laterally extending first waist edge 150 in the first waist region 140 and a longitudinally opposing and laterally extending second waist edge 152 in the second waist region 142. The disposable absorbent article 24 has a first side edge 154 and a laterally opposing second side edge 156, both side edges extending longitudinally between the first waist edge 150 and the second waist edge 152. The portion of the first side edge 154 in the first waist region 140 is designated 154a, the portion in the crotch region 144 is designated 154b, and the portion in the second waist region 142 is designated 154c. The corresponding portions of the second side edge 156 are designated 156a, 156b, and 156c, respectively.

The disposable absorbent article 24 preferably comprises a water-permeable topsheet 160, a water-impermeable backsheet 162, and an absorbent assembly or core 164, which may be disposed between the topsheet 160 and the backsheet 162 with the topsheet 160 attached to the backsheet 162. The topsheet 160 may be fully or partially elasticized or may be foreshortened. Exemplary structures including elasticized or foreshortened topsheets are described in greater detail in U.S. Pat. Nos. 4,892,536; 4,990,147; 5,037,416; and 5,269,775, among others.

The absorbent article 24 may include at least one elastic waist feature 170 that helps to provide improved fit and containment. The elastic waist feature 170 may be intended to elastically expand and contract to dynamically fit the wearer's waist. The elastic waist feature 170 may extend at least longitudinally outwardly from at least one waist edge (for example, edge 150) of the absorbent article 24 and generally forms at least a portion of the waist region (for example, region 140) of the absorbent article 24. Diapers are often constructed so as to have two elastic waist features 170, 172, one (170) positioned in the first waist region 140 and one (172) positioned in the second waist region 142. Other elastic waist feature constructions are described in U.S. Pat. Nos. 4,515,595; 4,710,189; 5,151,092; and 5,221,274.

The absorbent article 24 may include side panels 180, 182 attached to the backsheet 162. This construction may provide a more comfortable and contouring fit by initially conformably fitting the absorbent article 24 to the wearer, and sustaining this fit throughout the time of wear well past when the absorbent article 24 has been loaded with exudates, insofar as the elasticized side panels 180, 182 allow the sides of the absorbent article 24 to expand and contract. The side panels 180, 182 may also provide more effective application of the absorbent article 24 because even if the caretaker pulls one elasticized side panel 180 farther than the other (182) during application, the absorbent article 24 will “self-adjust” during wear. While the absorbent article 24 preferably has the side panels 180, 182 disposed in the second waist region 142, the absorbent article 24 may be provided with side panels disposed in the first waist region 140, or in both the front waist region 140 and the second waist region 142.

FIG. 17 illustrates the article illustrated in FIG. 16 configured to as it would be worn. The disposable absorbent article 24 may be sealed at the sides so as to be configured as illustrated in FIG. 17. However, the article 24 may instead include refastenable side seams that can be used to fasten the waist regions 140, 142 together. According to one exemplary embodiment, the waist regions 140, 142 may be fastened at the sides to apply the article like a diaper. According to an exemplary embodiment, illustrated in FIG. 17, the side seams may include fasteners 174 that can be used to configure the article like a pair of pull-on training pants or disposable pants.

As illustrated, the fasteners 174 may be disposed on the interior of the disposable absorbent article 24 in the second waist region 142 adjacent to the portion 154c of the first side edge 154 and adjacent to the portion 156c of the second side edge 156. The portion 154c of the side edge 154 is shown in an open condition, such as prior to closing and fastening or after being reopened. The portion 156c of the opposing side edge 156 is shown fastened, i.e., forming a pants configuration. In FIG. 17, the second waist region 142 overlaps the first waist region 140 when they are fastened together.

The fasteners 174 may be formed of any material and in any form that will releasably attach to the mating surface of the opposing waist region when pressed against it. For example, the primary fastening component may be a mechanical fastener that releasably engages with the mating surface, such as by means of a plurality of hooks engaging with loops formed by fibers in a nonwoven sheet. Alternatively, the primary fastening component may be an adhesive that releasably adheres to the mating surface. In fact, the fasteners may include cohesives, tape tabs, hook and loop fastening components, interlocking fasteners such as tabs & slots, buckles, buttons, snaps, and/or hermaphroditic fastening components. Exemplary surface fastening systems are disclosed in U.S. Pat. Nos. 3,848,594; 4,662,875; 4,846,815; 4,894,060; 4,946,527; 5,151,092; and 5,221,274, while an exemplary interlocking fastening system is disclosed in U.S. Pat. No. 6,432,098. The fastening system may also include primary and secondary fastening systems, as disclosed in U.S. Pat. No. 4,699,622. Additionally exemplary fasteners and fastener arrangements, the fastening components forming these fasteners, and the materials that are suitable for forming fasteners are described in U.S. Published Application Nos. 2003/0060794 and 2005/0222546 and U.S. Pat. No. 6,428,526.

Still other variations are also possible. For example, the fasteners 174 may be disposed on the interior of the article 24 in the first waist region 140 such that the first waist region 140 overlaps the second waist region 142 when they are fastened together. As another example, the fasteners 174 may be disposed on the exterior of the article 24 rather than on the interior. As a further example, the fasteners 174 may be used with a specific mating fastener surface particularly suited for cooperation with the fasteners 174 (for example, a loop layer that works with a hook fastener, or a layer particularly treated to provide a suitable contacting surface for a specific adhesive).

### Additional Variations to the Absorbent Article

In the alternative to the absorbent article 24 discussed above, various assemblies of topsheet 160, backsheet 162, and absorbent core 164 may be used. Additionally, the characteristics of the structures, the topsheet, for example, may be varied through the use of coatings, lotions, and the like. Further, in addition to the features described above, the disposable absorbent article 24 may include a variety of other features, such as slit openings, void spaces, leg cuffs, and the like to provide desired fit, containment, and aesthetic characteristics. Moreover, a transfer layer, which may also be referred to as an acquisition or distribution layer, or a sublayer may be disposed between the topsheet 160 and the core 164.

Thus, the topsheet, the backsheet, and the absorbent core may be assembled in the alternative in any of the well known configurations described in the following patent documents: U.S. Pat. Nos. 3,860,003; 5,151,092; 5,221,274; 5,554,145; 5,569,234; 5,580,411; and 6,004,306 (diapers) and U.S. Pat. Nos. 5,246,433; 5,569,234; 6,120,487; 6,120,489; 4,940,464; 5,092,861; 5,897,545; and 5,957,908 (pants).

The topsheet may be made from a wide range of materials, such as porous foams, reticulated foams, apertured plastic films, or woven or nonwoven materials of natural fibers (for example, wood or cotton fibers), synthetic fibers (for example, polyester or polypropylene fibers), or a combination of natural and synthetic fibers. If the topsheet includes fibers, the fibers may be spunbond, carded, wet-laid, meltblown, hydroentangled, or otherwise processed as is known in the art. One such material, including staple-length polypropylene fibers, is the P-8 material commercially available from Veratec, Inc., a Division of International Paper Company, of Walpole, Mass. Other alternative topsheets are described in U.S. Pat. Nos. 3,929,135; 4,324,246; 4,342,314; 4,463,045; and 5,006,394. Still other alternative topsheets may be made in accordance with U.S. Pat. Nos. 4,609,518 and 4,629,643. Films that may be used to make alternative topsheets may include DRI-WEAVE film commercially available from The Procter & Gamble Company of Cincinnati, Ohio and CLIFF-T film from Tredegar Corporation of Richmond, Va.

In certain embodiments, at least a portion of the topsheet is made of a hydrophobic material or is treated to be hydrophobic in order to isolate the wearer's skin from liquids contained in the absorbent core. If the topsheet is made of a hydrophobic material, preferably at least a portion of the upper surface of the topsheet is treated to be hydrophilic so that liquids will transfer through the topsheet more rapidly. The topsheet can be rendered hydrophilic by treating it with a surfactant or by incorporating a surfactant into the topsheet. Suitable methods for treating the topsheet with a surfactant include spraying the topsheet material with the surfactant and/or immersing the material into the surfactant. A more detailed discussion of such a treatment and hydrophilicity is contained in, for example, U.S. Pat. Nos. 4,988,344 and 4,988,345. A more detailed discussion of some suitable methods for incorporating a surfactant in the topsheet may be found in U.S. Statutory Invention Registration No. H1670. Alternatively, the topsheet may include an apertured web or film which is hydrophobic. This may be accomplished by eliminating the hydrophilizing treatment step from the production process and/or applying a hydrophobic treatment to the topsheet, such as a polytetrafluoroethylene compound like the SCOTCHGUARD product commercially available from 3M Corporation of Minneapolis, Minn., or a hydrophobic lotion composition. In such embodiments, it is preferred that the apertures be large enough to allow the penetration of aqueous fluids like urine without significant resistance.

Other materials may be applied or incorporated into the topsheet. For example, any portion of the topsheet may be coated with a lotion as is known in the art. Examples of suitable lotions include those described in U.S. Pat. Nos. 5,607,760; 5,609,587; 5,635,191; 5,643,588; 5,968,025 and 6,716,441. The lotion may function alone or in combination with another agent as the hydrophobizing treatment described above. The topsheet may also include or be treated with antibacterial agents, some examples of which are disclosed in PCT Publication No. WO 95/24173. Still other possibilities will be recognized.

The backsheet may be made from a thin plastic film, such as a thermoplastic film, having a thickness of about 0.012 mm (0.5 mil) to about 0.051 mm (2.0 mils). Exemplary backsheet films include the CPC2 film commercially available from Tredegar Corporation of Richmond, Va. Alternatively, the backsheet may be made from breathable materials, which materials permit vapors to escape from the absorbent article while still preventing bodily exudates from passing therethrough. Exemplary breathable materials may include woven webs, nonwoven webs, microporous films (such as ESPOIR NO film commercially available from Mitsui Toatsu Co. of Japan and EXAIRE film commercially available from Tredegar Corporation of Richmond, Va.), and monolithic films (such as HYTREL blend P18-3097 film commercially available from Clopay Corporation of Cincinnati, Ohio). Other alternative breathable materials may include composite materials, such as film-coated nonwoven webs or the composite materials described in PCT Publication No. WO 95/16746 and U.S. Pat. Nos. 5,938,648; 5,865,823; and 5,571,096.

The absorbent core may exhibit a wide variety of characteristics. For example, the core may be made in a wide variety of sizes and shapes (for example, rectangular, hourglass, “T”-shaped, asymmetric, etc.). Further, the absorbent core may be made from a wide variety of liquid-absorbent materials, such as those commonly used in disposable diapers and other absorbent articles, including comminuted wood pulp, which is generally referred to as airfelt. Other alternative absorbent materials may include creped cellulose wadding; meltblown polymers, including coform; chemically stiffened, modified or cross-linked cellulosic fibers; tissue, including tissue wraps and tissue laminates; absorbent foams; absorbent sponges; superabsorbent polymers; absorbent gelling materials; or any other known absorbent material or combinations of materials. The configuration and structure of the absorbent core may also be varied: for example, the absorbent core(s) or other absorbent structure(s) may have varying caliper zones, hydrophilic gradient(s), superabsorbent gradient(s), or lower average density and lower average basis weight acquisition zones. Exemplary structures for use as the absorbent core may include those in U.S. Pat. Nos. 4,610,678; 4,673,402; 4,834,735; 4,888,231; 5,137,537; 5,147,345; 5,342,338; 5,260,345; 5,387,207; and 5,625,222.

The backsheet may be joined to the topsheet, the absorbent core or any other element of the absorbent article. In this fashion, the backsheet and the topsheet may be joined directly to each other in some locations and indirectly together in other locations, for example, by directly joining them to one or more other elements of the absorbent article 24. The attachment may be formed by any of a variety of attachment methods or mechanisms. For example, the attachment methods or mechanisms may include a uniform continuous layer of adhesive, a patterned layer of adhesive, or an array of separate lines, spirals, or spots of adhesive. One possible attachment mechanism includes an open pattern network of filaments of adhesive, as in U.S. Pat. No. 4,573,986. Another suitable attachment mechanism includes several lines of adhesive filaments which are swirled into a spiral pattern, as in U.S. Pat. Nos. 3,911,173; 4,785,996; and 4,842,666. Other adhesives which may be used are the HL-1620 and HL-1358-XZP adhesives commercially available from H. B. Fuller Company of St. Paul, Minn. Further alternative attachment mechanisms may include heat bonding, pressure bonding, ultrasonic bonding, dynamic mechanical bonding, or any other suitable attachment mechanism or combinations of these attachment mechanisms.

The topsheet may comprise one or more apertures to ease penetration of exudates therethrough, such as urine and/or feces (solid, semi-solid, or liquid). In sizing the primary aperture, it is appropriate to consider that, if the primary aperture is too small, the waste may not pass through the aperture, either due to poor alignment of the waste source and the aperture location or due to fecal masses having a diameter greater than the aperture. Likewise, if the aperture is too large, the area of skin that may be contaminated by “rewet” from the article is increased. Typically, the aperture should have an area of between about 10 cm² and about 50 cm². The aperture preferably has an area of between about 15 cm² and 35 cm².

Moreover, the absorbent article may also include pockets for receiving and containing waste, spacers which provide voids for waste, barriers for limiting the movement of waste in the article, compartments or voids which accept and contain waste materials deposited in the absorbent article 24, and the like, or any combinations thereof. Examples of pockets and spacers for use in absorbent products are described in U.S. Pat. Nos. 5,514,121; 5,171,236; 5,397,318; 5,540,671; 6,168,584; 5,306,266; and 5,997,520. Examples of compartments or voids are disclosed in U.S. Pat. Nos. 4,968,312; 4,990,147; 5,062,840; and 5,269,755. Exemplary structures, including elasticized or foreshortened topsheets, to provide a void space between the topsheet and the core are described in more detail in U.S. Pat. Nos. 4,892,536; 4,990,147; 5,037,416; and 5,269,775. Examples of suitable transverse barriers are described in U.S. Pat. Nos. 5,554,142 and 5,653,703; and PCT Publication No. WO 94/14395. Examples of other structures especially suitable for management of low viscosity feces are disclosed in U.S. Pat. Nos. 5,941,864; 5,977,430 and 6,013,063.

The absorbent article may include leg cuffs which provide improved containment of liquids and other body exudates. Leg cuffs may also be referred to as leg bands, side flaps, barrier cuffs, or elastic cuffs. Suitable cuffs may be described in U.S. Pat. Nos. 3,860,003; 4,808,178; 4,909,803; 4,695,278; and 4,795,454. In some embodiments, it may be desirable to treat all or a portion of the leg cuffs with a lotion.

The absorbent article may also include a sublayer disposed between the topsheet and the backsheet. The sublayer may be any material or structure capable of accepting, storing or immobilizing bodily exudates. Thus, the sublayer may include a single material or a number of materials operatively associated with each other. Further, the sublayer may be integral with another element of the diaper or may be one or more separate elements joined directly or indirectly with one or more elements of the diaper. Further, the sublayer may include a structure that is separate from the core or may include or be part of at least a portion of the core.

Suitable materials for use as the sublayer may include large cell open foams, macro-porous compression resistant nonwoven highlofts, large size particulate forms of open and closed cell foams (macro and/or microporous), highloft nonwovens, polyolefin, polystyrene, polyurethane foams or particles, structures comprising a multiplicity of vertically oriented looped strands of fibers, absorbent core structures described above having punched holes or depressions, and the like. One embodiment of a sublayer includes XPL-7124 mechanical fastening loop landing element, having an uncompressed thickness of about 1.5 millimeters, commercially available from the 3M Corporation of Minneapolis, Minn. Another embodiment includes a 6 denier, crimped and resin-bonded nonwoven highloft, having a basis weight of 110 grams per square meter and an uncompressed thickness of 7.9 millimeters, commercially available from The Glit Company of Wrens, Ga. Other suitable absorbent and nonabsorbent sublayers are described in U.S. Pat. Nos. 6,680,422 and 5,941,864. Further, the sublayer, or any portion thereof, may include or be coated with a lotion or other known substances to add, enhance or change the performance or other characteristics of the element.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description are, in relevant part, incorporated herein by reference; the citation of any document is not to be construed as an admission that it is prior art with respect to the present invention. To the extent that any meaning or definition of a term in this disclosure conflicts with any meaning or definition of the term in a document incorporated by reference, the meaning or definition assigned to the term in this disclosure shall govern for this disclosure.

While particular embodiments of the present disclosure have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A kit, comprising:
a package;
an absorbent article having a topsheet, a backsheet, and an absorbent core disposed between the topsheet and the backsheet, the absorbent article being disposed in the package in a storage state and removable from the package in an operative state; and
a diagnostic device being disposed on the package in the storage state and detachable from the package in the operative state.

2. The kit according to claim 1, wherein the diagnostic device is a temperature sensor.

3. The kit according to claim 1, wherein the package includes a wall, and the diagnostic device is defined, at least in part, by a region of the wall.

4. The kit according to claim 3, wherein the package includes a perforation disposed about the region to permit the region to be detached from the remainder of the wall.

5. The kit according to claim 4, wherein the package includes a closure element, the closure element being defined by the region.

6. The kit according to claim 3, wherein a temperature-sensitive material is disposed on the region of the wall.

7. The kit according to claim 6, wherein the temperature-sensitive material is selected from the group of temperature-sensitive materials consisting of thermochromic liquid crystal materials, thermochromic inks, thermochromic dyes and combinations thereof.

8. The kit according to claim 1, further comprising a plurality of absorbent articles having a topsheet, a backsheet, and an absorbent core disposed between the topsheet and the backsheet, the plurality of absorbent articles being disposed in the package in a storage state and removable from the package in an operative state

9. A kit, comprising:
a package;
an absorbent article having a topsheet, a backsheet, and an absorbent core disposed between the topsheet and the backsheet, the absorbent article being disposed in the package in a storage State and removable from the package in an operative state; and
a supply selected from the group of supplies consisting of a therapeutic agent and a therapeutic device, the supply being disposed on or in the package in the storage state and removable from the package in the operative state.

10. The kit according to claim 9, the therapeutic agent including a compound for treating a fever.

11. The kit according to claim 9, the therapeutic device including a hot pack or a cold pack.

12. The kit according to claim 9, wherein the absorbent article and the supply are distinct and separate items, disposed in the package in the storage state.

13. The kit according to claim 9, wherein the package has a wall, and the supply is joined to the wall.

14. The kit according to claim 9, further comprising a plurality of absorbent articles having a topsheet, a backsheet, and an absorbent core disposed between the topsheet and the backsheet, the plurality of absorbent articles being disposed in the package in a storage state and removable from the package in an operative state.

15. A kit, comprising:
a package;
an absorbent article having a topsheet, a backsheet, and an absorbent core disposed between the topsheet and the backsheet, the absorbent article being disposed in the package in a storage state and removable from the package in an operative state;
a supply selected from the group of supplies consisting of a therapeutic agent and a therapeutic device, the supply being disposed on or in the package in the storage state and removable from the package in the operative state; and
a diagnostic device being disposed on or in the package in the storage state and removable from the package in the operative state.

16. The kit according to claim 15, wherein the diagnostic device is a temperature sensor.

17. The kit according to claim 16, wherein the package includes a wall, and the diagnostic device is defined, at least in part, by a region of the wall.

18. The kit according to claim 16, wherein the temperature sensor is formed with the article.

19. The kit according to claim 18, wherein the temperature sensor is detachable from the article.

20. The kit according to claim 16, wherein the temperature sensor is formed of a temperature-sensitive material selected from the group of temperature-sensitive materials consisting of thermochromic liquid crystal materials, thermochromic inks, thermochromic dyes and combinations thereof.
